(19) United States
(12) Patent Application Publication
(10) Pub. No.: US 2008/0267978 A1
(43) Pub. Date: Oct. 30, 2008

(54) Anti-angiogenic targets for cancer therapy

(76) Inventor: Mary Zutter, Nashville, TN (US)

Correspondence Address: STITES & HARBISON PLLC, 401 COMMERCE STREET, SUITE 800, NASHVILLE, TN, 37219, US

(21) Appl. No.: 11/892,964

(22) Filed: Aug. 28, 2007

Related U.S. Application Data
(60) Provisional application No. 60/840,447, filed on Aug. 28, 2006.

(51) Int. Cl. A61K 39/395; A61P 35/00

(52) U.S. Cl. 424/174.1; 424/172.1

(57) ABSTRACT

The use of inhibitory anti-α2β1 integrin antibodies to inhibit tumor neoangiogenesis, slow tumor growth, treat abnormal angiogenesis, treat integrin-mediated disorders and inhibit endothelial cell proliferation.

Tumor initiation and progression involve complex interactions between tumor cells and their microenvironment [Hanahan D, Weinberg R A: The hallmarks of cancer. Cell 2000, 100(1):5770]. The tumor microenvironment consists of the three-dimensional extracellular matrix (ECM) surrounding tumor cells, as well as host cells such as endothelial cells, pericytes, fibroblasts and inflammatory cells [Hanahan D, Weinberg R A: The hallmarks of cancer. Cell 2000, 100(1):5770; Vogelstein B, Kinzler K W: Cancer genes and the pathways they control. Nat Med 2004, 10(8): 789-799; DeClerck Y A, Mercurio A M, Stack M S, Chapman H A, Zutter M M, Muschel R J, Raz A, Matrisian L M, Sloane B F, Noel A et a/: Proteases, extracellular matrix, and cancer: a workshop of the path B study section. Am] Patho/2004, 164(4): 1131-1139; Coussens L M, Fingleton B, Matrisian L M: Matrix metalloproteinase inhibitors and cancer: trials and tribulations. Science 2002, 295(5564): 2387-2392; Matrisian L M, Sledge G W, Jr., Mohla S: Extracellular proteolysis and cancer: meeting summary and future directions. Cancer Res 2003, 63(19): 6105-6109; Hanahan D, Lanzavecchia A, Mihich E: Fourteenth Annual Pezcoller Symposium: the novel dichotomy of immune interactions with tumors. Cancer Res 2003, 63(11):3005-3008; 7. Cunha G R, Matrisian LM: It's not my fault, blame it on my microenvironment. Differentiation 2002, 70(9-10):469-472].

The ECM provides not only scaffolding, but also signals to tumor cells through ECM receptors such as integrins. Moreover, it serves as a reservoir for growth factors, cytokines, and a pool of “silent” molecules such as the “statins” and perlecan that directly affect tumor angiogenesis after degradation by specific proteolytic enzymes.

Members of the integrin family of extracellular matrix receptors are expressed on many tumor types as well as on cells of the tumor microenvironment. Different integrins have been implicated to play distinct roles in tumor initiation and progression, as well as important roles in angiogenesis [Hynes R O: A reevaluation of integrins as regulators of angiogenesis Nat Med 2002, 8(9):918-921]. The finding that integlins are readily accessible drug targets for therapy [Hynes R O: A reevaluation of integrins as regulators of angiogenesis. Nat Med 2002, 8(9):918-921; Jain RK: Molecular regulation of vessel maturation. Nat Med 2003, 9(6): 685-693; McDonald D M, Teicher B A, Stetler-Stevenson W, Ng S S, Figg W D, Folkman J, Hanahan D, Auerbach R, O'Reilly M, Herbst R et a/: Report from the society for biological therapy and vascular biology faculty of the NCI workshop on angiogenesis monitoring.] Immunother 2004, 27(2):161175; Kerbel R, Folkman J: Clinical translation of angiogenesis inhibitors. Nat Rev Cancer 2002, 2(10):727-739] makes these receptors attractive targets for antiangiogenic therapy. Several integrins, specifically the avβ3 and avβ3, have been implicated in angiogenesis, although their role remains controversial. In this context, it has been shown that integrin avβ3 is expressed on the tip of sprouting vessels and can promote angiogenesis by binding matrix metalloproteinase (MMP)2, thus facilitating ECM degradation and new blood vessel formation [Brooks P C, Stromblad S, Sanders L C, von Schalscha T L, Aimes R T, StetlerStevenson W G, Quigley J P, Cheresh D A: Localization of matrix metalloproteinase MMP-2 to the surface of invasive cells by interaction with integrin alpha v beta 3. Ce1/1996, 85(5):683-693]. Inhibition of integrin α2β3 by blocking antibodies has been shown to suppress neovascularization and tumor growth, suggesting that this receptor may be a critical modulator of angiogenesis [Brooks P C, Montgomery A M, Rosenfeld M, Reisfeld R A, Hu T, Klier G, Cheresh D A: Integrin alpha v beta 3 antagonists promote tumor regression by inducing apoptosis of angiogenic blood vessels. Ce1/1994, 79(7): 1157-1164; Brooks P C, Clark R A, Cheresh D A: Requirement of vascular integrin alpha v beta 3 for angiogenesis. Science 1994, 264(5158):569-571; Eliceiri B P, Cheresh D A: The role of alphav integrins during angiogenesis: insights into potential mechanisms of action and clinical development.] Clin Invest 1999, 103(9): 1227-1230].

The integrins play critical roles in tumor-host interactions. Several integrins, including the α1β1 and α2β1 integrin receptors for collagens, have been implicated in angiogenesis. Genetic deletion of the α1β1 integrin supported the concept that the α1β1 integrin was pro-angiogenic. In contrast, genetic deletion of the α2β1 integrin leads to increased tumor angiogenesis and normalization of the vasculature. The findings supported by the research reported herein that lack of the α2β1 integrin in the host microenvironment shifts the angiostatic balance in favor of angiogenesis demonstrates for the first time that expression of the α2β1 integrin is anti-angiogenic and regulates tumor vasculature morphogenesis in vivo. These findings shift the paradigm and demonstrate that integrins control vasculature differentiation and not just endothelial cell proliferation and survival.

The role of a α1β1 and α2β1 integrins, the two major collagen receptors, in angiogenesis also has been evaluated, although much less extensively in vivo [Senger D R, Claffey K P, Benes J E, Perruzzi C A, Sergiou A P, Detmar M: Angiogenesis promoted by vascular endothelial growth factor: regulation through alpha1beta1 and alpha2beta1 integrins. Proc Natl Acad Sci USA 1997, 94(25):13612-13617; Senger D R, Perruzzi C A, Streit M, Koteliansky V E, de Fougerolles A R, Detmar M: The alpha(1)beta(1) and alpha(2)beta(1) integrins provide critical support for vascular endothelial growth factor signaling, endothelial cell migration, and tumor angiogenesis. Am] Pathol 2002, 160(1): 195204; Whelan M C, Senger D R: Collagen I initiates endothelial cell morphogenesis by inducing actin polymerization through suppression of cyclic AMP and protein kinase A.] Biol Chem 2003, 278(1):327-334; Sweeney S M, Dilullo G, Slater S J, Martinez J, lozzo R V, lauer-Fields J I, Fields G B, San Antonio JD: Angiogenesis in collagen I requires alpha2beta1 ligation of a GFP*GER sequence and possibly p38 MAPK activation and focal adhesion disassembly.] Biol Chem 2003, 278(33):3051630524; Hong Y K, lange-Asschenfeldt B, Velasco P, Hirakawa S, Kunstfeld R, Brown I F, Bohlen P, Senger D R, Detmar M: VEGF-A promotes tissue repairassociated lymphatic vessel formation via VEGFR-2 and the alpha1beta1 and alpha2beta1 integrins. Faseb J 2004, 18(10): 11111113; Liu Y, Senger D R: Matrix-specific activation of Src and Rho initiates capillary morphogenesis of endothelial cells. Faseb J 2004, 18(3):457-468; Pozzi A, Moberg P E, Miles L A, Wagner S, Soloway P, Gardner HA: Elevated matrix metalloprotease and angiostatin levels in integrin alpha 1 knockout mice cause reduced tumor vascularization. Proc Nat/Acad Sci USA 2000, 97(5):2202-2207; Pozzi A, Wary K K, Giancotti F G, Gardner HA: Integrin alpha1beta1 mediates a unique collagen-dependent proliferation pathway in vivo.) Cell Sio/1998, 142(2): 587-594]. Senger and colleagues argued that receptors for collagens, the predominant ECM molecules within the tumor microenvironment, are critical for the development of new vessels, based on the finding that treatment of human umbilical vein endothelial cells with anti-α1 and/or anti-α2 integrin antibodies, in combination, inhibited in vitro endothelial cell adhesion, spreading on collagen I gels and vascular endothelial growth factor (VEGF)-stimulated chemotaxis. Moreover, use of these antibodies in vivo prevented VEGF-driven angiogenesis, providing additional evidence for the proangiogenic role of both α1β1 and α2β1 integrins [Chen J, Diacovo T G, Grenache D G, Santoro S A, Zutter M M: The alpha(2) integrin subunit-deficient mouse: a multifaceted phenotype including defects of branching morphogenesis and hemostasis. Am) Patho/2002, 161(1):337-344].

Genetic deletion of the α1β1 integrin supported the concept that the α1β1 integrin was pro-angiogenic. Although α1-null mice develop a normal cardiovascular system during embryogenesis, α1-deficient mice injected with syngeneic tumors demonstrate decreased tumor growth and decreased tumor angiogenesis. Decreased angiogenesis in these mice is due to the increased levels of circulating angiostatin, as a result of increased expression and activation of MMP7 and MMP9. These in vivo data are consistent with the α1β1 integrin serving a proangiogenic function.

The integrin α2β1 (Very late antigen 2; VLA-2) is expressed on a variety of cell types including platelets, vascular endothelial cells, epithelial cells, activated monocytes/macrophages, fibroblasts, leukocytes, lymphocytes, activated neutrophils and mast cells. (Hemler, Annu Rev Immunol8:365:365-400 (1999); Wu and Santoro, Dev. Dyn. 206:169-171 (1994); Edelson et. al., Blood. 103(6):2214-20 (2004); Dickeson et al, Cell Adhesion and Communication, 5: 273-281 (1998)). The most typical ligands for α2β1 include collagen and laminin, both of which are found in extracellular matrix. Typically the I-domain of the α2 integrin binds to collagen in a divalent-cation dependent manner whereas the same domain binds to laminin through both divalent-cation dependent and independent mechanisms. (Dickeson et al, Cell Adhesion and Communication. 5:273-281 (1998)) The specificity of the α2β1 integrin varies with cell type and serves as a collagen and/or laminin receptor for particular cell types, for example α2β1 integrin is known as a collagen receptor for platelets and a laminin receptor for endothelial cells. (Dickeson et al, J. Biol. Chem., 272: 7661-7668 (1997)) Echovirus-I, decorin, E-cadherin, matrix metalloproteinase I (MMP-I), endorepellin and multiple collectins and the C1q complement protein are also ligands for α2β1 integrin. (Edelson et al., Blood 107(1):143-50 (2006)). The α2β1 integrin has been implicated in several biological and pathological processes including collagen-induced platelet aggregation, cell migration on collagen, cell-dependent reorganization of collagen fibers as well as collagen-dependent cellular responses that result in increases in cytokine expression and proliferation, (Gendron, J. Biol. Chem. 278:48633-48643 (2003); Andreasen et al., J. Immunol. 171:2804-2811 (2003); Rao et al., J. Immunol. 165(9):4935-40 (2000)), aspects of T-cell, mast cell, and neutrophil function (Chan et. al., J. Immunol. 147:398-404 (1991); Dustin and de Fougerolles, Curr Opin Immunol 13:286-290 (2001), Edelson et. al, Blood. 103(6):2214-20 (2004), Werr et al, Blood 95:1804-1809 (2000), aspects of delayed type hyersensitivity contact hypersensitivity and collagen-induced arthritis (de Fougerolles et. al, J. Clin. Invest. 105:721-720 (2000); Kriegelstein et al, J. Clin. Invest. 110(12): 1773-82 (2002)), mammary gland ductal morphogenesis (Keely et. al, J. Cell Sci. 108:595-607 (1995); Zutter et al, Am. J. Pathol 155(3):927-940 (1995)), epidermal wound healing (Pilcher et. al., J. Biol Chem. 272:181457-54 (1997)), and processes associated with VEGF-induced angiogenesis (Senger et al, Am. J. Pathol. 160(1): 195-204 (2002)) circulation into tissues in response to inflammatory stimuli, including migration, recruitment and activation of proinflammatory cells at the site of inflammation (Eble J. A., Curro Pharo Des. 11(7):867-880 (2005)). Some antibodies that block α2β1 integrin were reported to show impact on delayed hypersensitivity responses and efficacy in a murine model of rheumatoid arthritis and a model of inflammatory bowel disease (Kriegel stein et al, J. Clin. Invest. 110(12): 1773-82 (2002); de Fougerolles et al, 1. Clin. Invest. 105:721-720 (2000) and were reported to attenuate endothelial cell proliferation and migration in vitro (Senger et al, Am. J. Pathol. 160(1):195-204 (2002), suggesting that the blocking of α2β1 integrin might prevent/inhibit abnormal or higher than normal angiogenesis, as observed in various cancers.

α2β1 integrin is the only collagen-binding integrin expressed on. platelets and has been implicated to play some role in platelet adhesion to collagen and hemostasis (Giruner et al, Blood 102:4021-4027 (2003); Nieswandt and Watson, Blood 102(2):449-461 (2003); Santoro et al, Thromb. Haemost. 74:813-821 (1995); Siljander et al, Blood 15:13331341 (2004); Vanhoorelbeke et al, Curro Drug Targets Cardiovasc. Haemato J. Disord. 3(2): 125-40 (2003)). In addition, platelet α2β1 may play a role in the regulation of the size of the platelet aggregate (Siljander et al, Blood 103(4):1333-1341 (2004)). α2β1 integrin has also been shown as a lamininbinding integrin expressed on endothelial cells (Languino et al,] Cell Bio. 109:2455-2462 (1989)). Endothelial cells are thought to attach to laminin through an integrin-mediated mechanism. however it has been suggested that the α2 I domain may function as a ligand-specific sequence involved in mediating endothelial cell interactions (Bahou et al, Blood. 84(11):3734-3741 (1994)).

The anti-human α2β1 integrin blocking antibody BHA2.1 was first described by Hangan et al, (Cancer Res. 56:3142-3149 (1996)). Other anti-α2β1 integrin antibodies are known and have been used in vitro, such as the commercially available antibodies AK7 (Mazurov et al, Thromb. Haemost. 66(4): 494-9 (1991), P I E6 (Wayner et al, J. Cell Biol. 107(5):1881-91 (1988)), 10G11 (Giltay et al, Blood 73(5):1235-41 (1989) and A2-11E10 (Bergelson et aj, Cell Adhes. Commun. 2(5):455-64 (1994). Hangan et al, (Cancer Res. 56:3142-3149 (1996)) used the BHA2.1 antibody in vivo to study the effects of blocking α2β1 integrin function on the extravasation of human tumor cells in the liver, and the ability of these tumor cells to develop metastatic foci under antibody treatment. The Hal/29 antibody (Mendrick and Kelly, Lab Invest. 69(6):690-702 (1993)), specific for rat and murine a, 2p I integrin, has been used in vivo to study the upregulation of α2β1 integrin on T cells following LCMV viral activation (Andreasen et al, J. Immunol. 171:2804-2811 (2003)), to study SRBC-induced delayed type hypersensitivity and FITC-induced contact type-hypersensitivity responses and collagen-induced arthritis (de Fougerolles et. al, l. Clin. Invest. 105:721-720 (2000)), to study the role of α2β1 integrin in VEGF regulated angiogenesis (Senger et al, Am. J. Pathol. 160(1): 195204 (2002); Senger et al, PNAS 94(25): 13612-7 (1997)), and to study the role of α2β1 integrin in PMN locomotion in response to platelet activating factor (PAP) (Werr et al, Blood 95:1804-1809 (2000)).

Although the role of the α2β1 integrin in vitro has been well defined, little is known regarding the expression and function of the α2β1 integrin in the tumor microenvironment in vivo.

### SUMMARY OF THE INVENTION

An embodiment of the invention relates to the use of inhibitory anti-α2β1 integrin antibodies to inhibit tumor neoangiogenesis, slow tumor growth and inhibit endothelial cell proliferation.

### BRIEF DESCRIPTION OF THE DRAWINGS

FIG. 1 shows the growth of syngeneic, B16F10 melanoma in α2β1 integrin-deficient mice.

FIG. 2 shows the vascular perfusion and tumor necrosis in tumors in α2-null and wild type mice.

FIG. 3 shows the α2β1 integrin expression and matrix-independent endothelial cell proliferation in vivo and in vitro.

FIG. 4 demonstrates VEGFR1 but not VEGFR2 expression is upregulated on α2-null endothelial cells in vitro and in vivo.

FIG. 5 demonstrates that tumor angiogenesis is increased in spontaneous MMTV-PyMT-induced mammary carcinomas arising in the α2β1 integrin deficient-mouse.

FIG. 6 demonstrates that angiogenesis is inhibited in wild type mice treated with an inhibitory anti α2β1 integrin antibody.

FIG. 7 depicts a model of α2β1 integrin regulated-neoangiogenesis.

FIG. 8 depicts the expression of the α2β1 integrin by resting and tumor vessels.

FIG. 9 demonstrates that the expression of the α1β1 integrin is not upregulated on α2-null endothelial cells in vitro or in vivo.

### DETAILED DESCRIPTION OF THE INVENTION

The present invention is predicated on (1) the discovery that the α2β1 integrin plays a major role in maintaining a pro-angiogenic state in vivo (the α2β1 integrin is either not expressed or expressed at undetectable levels on resting endothelial cells, but the integrin is upregulated on endothelium within the tumor microenvironment); and (2) maintenance of a balance between the α2β1 integrin and the α1β1 integrin serves to control the angiostatic set point. Neither the α2β1 or α1β1 integrin is required for developmental angiogenesis. In the resting state, endothelial cells express extremely low levels of both the α2β1 and the α1β1 integrin. However, under circumstances of pathologic angiogenesis, such as the tumor microenvironment, expressions of both the α2β1 and the α1β1 integrins is rapidly upregulated in wild type animals. These two integrins are not redundant but have distinct roles in angiogenesis. The α1β1 integrin provides pro-proliferative signals. In contrast, signals from the α2β1 integrin are anti-proliferative and serve to regulate vascular morphogenesis, suggesting that the two receptors serve a homeostatic balance. Part of the homeostatic balance involves down-regulation of the VEGFR1 expression by the α2β1 integrin. In the α2β1 integrin-deficient mouse, for example, the α2β1 integrin-dependent anti-proliferative signals are released and VEGR1 is significantly upregulated on the tumor vessels but not other vessels within the animal. Neoangiogenesis is unchecked and vascular normalization occurs.

On the other hand, when inhibitory anti-α2β1 integrin antibodies are introduced into the system, the α2β1 integrin is ligated and antiproliferative signals emanating from the integrin are augmented leading to an inhibition of endothelial cell proliferation and a marked inhibition of angiogenesis.

As noted above, the integrin family of extracellular matrix receptors plays critical roles in the tumor microenvironment. To define the contributions of α2β1 integrin in pathologic angiogenesis, tumor growth and tumor angiogenesis was compared in wild type mice and α2β1 integrin-deficient mice; providing evidence that α2β1 integrin deficiency leads to increased tumor angiogenesis, vascular normalization and accelerated tumor growth. Up-regulated tumor angiogenesis is due to increased α2-null endothelial cell proliferation both in vitro and in vivo. In contrast to α2β1 integrin deletion, inhibitory anti-α2β1 integrin antibody inhibited tumor angiogenesis, tumor growth, and inhibited endothelial cell proliferation. This data suggest for the first time that the α2β1 integrin negatively regulates angiogenesis and vascular normalization.

To define the contributions of α2β1 integrin expression to angiogenesis, the molecular regulation of neoangiogenesis was compared in wild type mice and mice lacking expression of the α2β1 integrin. The evidence shows that, unlike the α1-null mice, α2-null mice exhibit increased tumor angiogenesis, increased tumor vessel stability with improved perfusion and consequent increased tumor growth and decreased tumor necrosis when challenged with syngeneic B16F10 melanoma cells. In addition, increased tumor angiogenesis in the α2β1 integrin-deficient animals was also observed in spontaneous MMTV-PyMT transgene-induced breast tumors [Shan S, Robson N O, Cao Y, Qiao T, Li C Y, Kontos C D, Garcia-Blanco M, Dewhirst M W: Responses of vascular endothelial cells to angiogenic signaling are important for tumor cell survival. FasebJ 2004, 18(2): 326-328]. Enhanced tumor angiogenesis is due to increased integrin α2-null endothelial cell proliferation both in vitro and in vivo. Moreover, it is shown herein that increased expression of vascular endothelial cell growth factor receptor (VEGFR)-1 on α2-null endothelial cells is at least in part responsible for their increased proliferation. In contrast to the data obtained in the α2-null mouse, the data also demonstrate that inhibitory anti-α2β1 integrin antibodies inhibit tumor neoangiogenesis, slow tumor growth and inhibit endothelial cell proliferation.

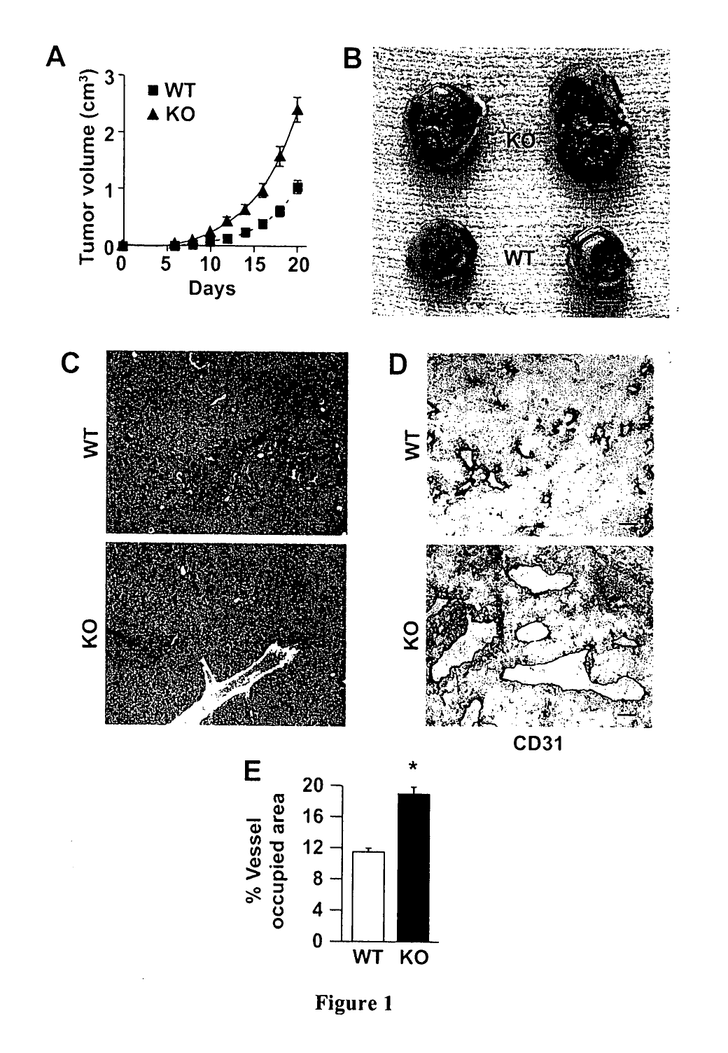

The data also suggest that vascular normalization in the absence of the α2β1 integrin results from upregulation of angiogenic growth factor receptors that control vessel morphogenesis in addition to proliferation. The integrins may serve as master regulators of neoangiogenesis by regulating the levels of VEGFR1 and VEGFR2 required for normal vascular development. The data further suggest that the α2β1 integrin negatively regulates endothelial cell proliferation within the tumor microenvironment. In the presence of inhibitory antibodies our data suggest that the antiproliferative signals downstream of α2β1 integrin ligation augment the antiproliferative activity. To determine whether α2β1 integrin expression by cells of the host microenvironment plays a role in tumor growth, wild type and α2-null mice (on a pure C57/BL6 background) were injected subcutaneously with syngeneic, B16F10 melanoma cells and monitored for palpable tumors every two days for 21 days. As shown in FIG. 1A, tumors grew in both wild type and α2-null mice. However, tumor growth was more rapid in α2-null mice in comparison to wild-type mice. Tumor size was significantly larger in the α2-null mice than wild type controls at all time points from day 6 to day 21 (FIGS. 1A and B).

After 21 days the tumors were excised, and tumor morphology was evaluated using paraffin-embedded, hematoxylin and eosin (H&E)-stained sections. As shown in FIG. 1C, tumors derived from both wild type and α2-null mice were composed of sheets of tumor cells with focal melanin pigmentation and minimal inflammation. Tumor vessels in the α2-null mice were larger and irregular in shape when compared to vessels of wild type littermates. Motivated by the altered vascular morphology observed, tumor vessel volume and vessel number were subsequently evaluated. Immunohistochemical staining with anti-CD31 antibody accentuated vessel morphology (FIG. 1D) and highlighted the significant increase (65%) in the total area occupied by vessels and in the average vessel size in tumor sections from α2-null mice compared to wild type mice (FIG. 1E).

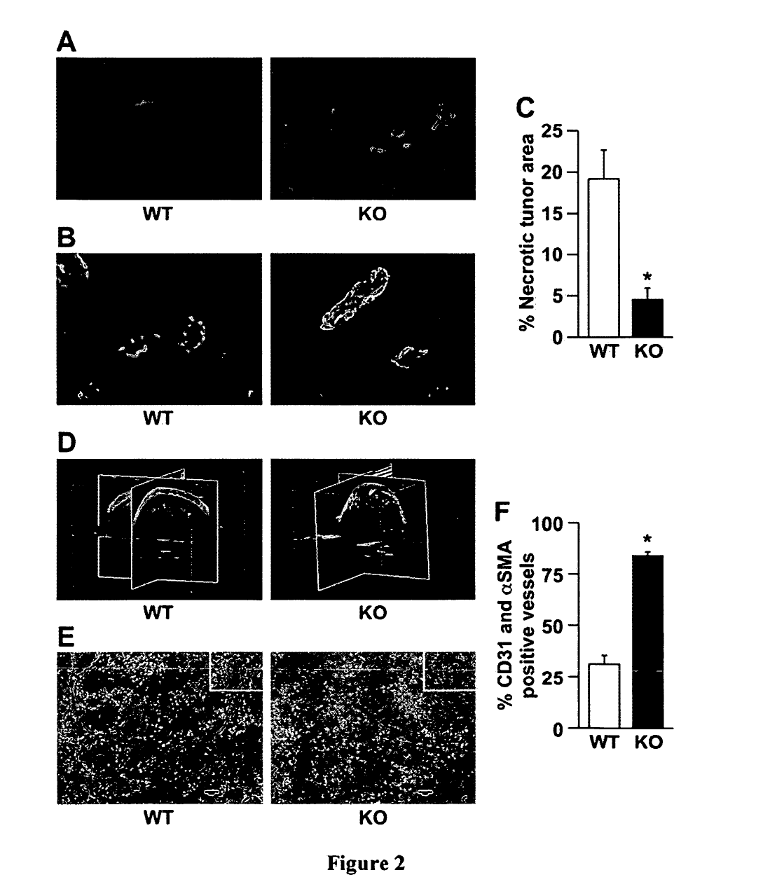

Based on vessel morphology of tumors in the α2-null mouse, it was hypothesized that the vessels were dilated, leaky and therefore dysfunctional. To address this issue, mice were injected with TRITC-dextran and its retention and/or extravasation in tumor blood vessels was evaluated. Consistent with previous literature, extravasation of TRITC-dextran from wild type vessels was notable (FIG. 2A) [26] [27]. In contrast, tumor vessels in the α2-null mouse retained the TRITC-dextran within the vasculature (FIG. 2A). The appearance of the α2-null vessels was therefore not due to vessel leakiness.

The process of vascular normalization requires vascular stabilization and is dependent upon the effective recruitment of pericytes. A role for integrin receptors in vascular normalization has not been previously described. To determine whether alterations in vessel leakage were due to altered recruitment of pelicytes in the wild type versus α2-null animals, pericyte recruitment was evaluated by co-staining tumor sections with anti a-SMA and anti-CD31 antibodies. Consistent with published data [26, 28] the majority of the vessels in the wild type mouse lacked pericytes and only occasional tumor vessels were encircled by a-SMA positive pericytes (FIG. 2B). In contrast, the vast majority of α2-null vessels were completely encircled by pericytes (FIG. 2B). 84% of CD31-positive tumor vessels were surrounded by aSMA positive pericytes in the α2-null mice, while only 31% of tumor vessels in wild type mice were encircled by pericytes (FIG. 2C). These results demonstrate that lack of α2β1 integrin expression favors vascular stabilization with augmented pericyte recruitment.

It was hypothesized that increased numbers of large, stabilized vessels would permit for increased blood flow to the tumor vascular bed. To better define vascular perfusion, the amplitude of blood flow within the tumor was determined by Power Doppler. Power Doppler, now the preferred method to characterize blood flow, measures the relative number of flowing cells and is significantly more sensitive to small vessels (“50 microns) with low flow than other techniques such as frequency Doppler. As shown in FIG. 2D, there was a marked increase in blood flow to tumors in the α2-null mice in comparison to wild type mice at 14 days after tumor injection. Total blood flow to the tumors in α2-null mice was 7.68+/−1.24% and significantly increased in comparison to blood flow in wild type animals, 2.5+/0.43%. As expected from the dramatic differences in tumor blood flow, the area of necrotic tumor was significantly decreased in the α2β1 integrin-deficient mice (4.3% of tumor area) when compared to their littermate controls (19.2% of tumor area) (FIGS. 2E and F). These results demonstrate increased numbers of stabilized vessels provide increased blood flow to the tumor bed in the α2-null mice, a phenotype suggestive of vascular normalization.

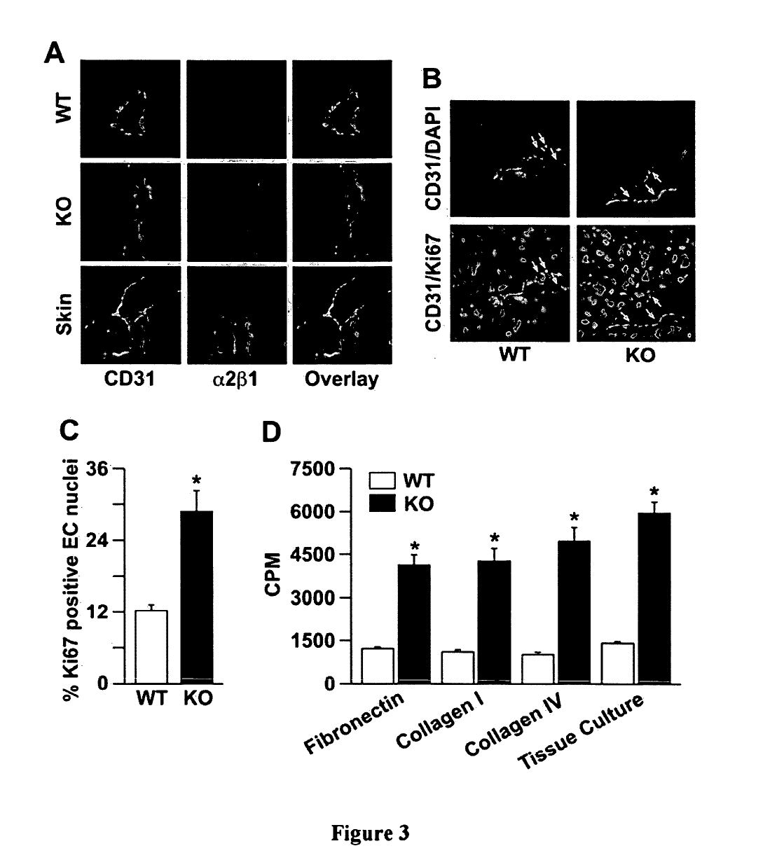

The α2β1 integrin is expressed at low or undetectable levels on many endothelial cells including the microvascular endothelial cells of the dermis [Senger D R, Claffey K P, Benes J E, Perrzzi C A, Sergiou A P, Detmar M: Angiogenesis promoted by vascular endothelial growth factor: regulation through alpha1beta1 and alpha2beta1 integrins. Proc Natl Acad Sci USA 1997, 94(25):13612-13617; Senger D R, Perruzzi C A, Streit M, Koteliansky V E, de Fougerolles A R, Detmar M: The alpha(1)beta(1) and alpha(2)beta(1) integrins provide critical support for vascular endothelial growth factor signaling, endothelial cell migration, and tumor angiogenesis. Am J Pathol 2002, 160(1): 195204]. Expression of the α2 integrin subunit is upregulated on dermal microvascular endothelial cells in vitro within 4-8 hours of stimulation with VEGF165 [16]. Since expression of the α2β1 integrin in vivo on tumor vessels has not been described, the expression of the α2β1 integrin on tumor vessels was examined by co-staining with anti-integlin a2 and anti-CD31 antibodies. As shown in FIG. 3A and Supplemental FIG. 1, the α2β1 integrin was expressed at high levels by CD31 positive endothelial cells in the wild type tumors, but not in tumors from the α2-null animals, a negative control for immunofluorescence staining. There was no discernable expression of the α2β1 integrin by the smooth muscle cells/pericytes. The tumor vasculature did not express the lymphatic marker LYVE-1, therefore the vessels were not of lymphatic origin (data not shown). Expression of the α2β1 integrin on neovascular tumor endothelial cells was markedly upregulated in comparison to its expression on quiescent endothelial cells in other tissues including the skin, heart, kidney, liver, lung, and spleen (FIG. 3A and Supplemental FIG. 1).

The increased number and size of the vessels in the α2-deficient mouse suggested that there were increased numbers of endothelial cells. Therefore, proliferative activity of tumor endothelial cells was determined by co-staining tumor sections with anti-Ki67 and anti-CD31 antibodies. Endothelial cell nuclei were defined by DAPI staining of CD31 positive cells. 28.7% of α2-null endothelial cells, but only 12.4% of wild type endothelial cells coexpressed CD31 and Ki67 (FIGS. 3B and C). Therefore the α2-deficient endothelial cells demonstrate increased proliferation in vivo.

In summary, it has been demonstrated a that lack of α2β1 integrin leads to increased tumor vessel size, number, and stability, hallmarks of normalized vessels.

Moreover, increased endothelial cell proliferation was observed in tumors grown in the α2-null microenvironment. The increased vascularization with improved vascular stability resulted in improved vascular perfusion, decreased tumor necrosis and increased tumor growth in the α2-null hosts.

It was further hypothesized that the increased tumor angiogenesis and endothelial cell proliferation in vivo were due to alterations in endothelial cell function. To define the functional role of α2β1 integrin on endothelial cells, primary pulmonary microvascular endothelial cells were isolated from wild type and α2β1 integrin-deficient mice. Endothelial cells were plated on type I or type IV collagen (both α2β1 integrin-dependent ligands), fibronectin (an α2β1 integrin-independent ligand), or tissue culture plastic. Cell proliferation was then evaluated by measuring 3H-thymidine incorporation. α2-null endothelial cells showed a four to five fold higher proliferative index compared to their wild type counterparts (FIG. 3D). Surprisingly, α2-null cells exhibited increased proliferation not only on the collagen I and IV substrates, but also on either fibronectin or tissue culture plastic. The proliferative advantage was therefore matrix-independent.

The matrix-independent proliferation suggested that α2-null endothelial cells were intrinsically more proliferative. Animals lacking expression of the α1β1 integrin, the other major collagen receptor, demonstrated decreased tumor angiogenesis and showed decreased endothelial cell proliferation under similar experimental conditions [Pozzi A, Wary K K, Giancotti F G, Gardner H A: Integrin alpha1beta1 mediates a unique collagen-dependent proliferation pathway in vivo.) Cell Sio/1998, 142(2): 587-594]. The augmented tumor angiogenesis in the α2β1 integrin-deficient mice suggested that over-expression of the α1β1 integrin may serve a compensatory role. Expression of α1β1 integrin subunit mRNA by wild type and α2-null primary pulmonary endothelial cells was determined by quantitative (q)RT-PCR. As shown in Supplemental FIG. 2, no significant differences in the level of the α1β1 integrin subunit mRNA were observed. Expression of the α1β1 integrin on tumor vessels was also evaluated by immunofluorescence analysis. Consistent with the qRT-PCR data there were no detectable differences in the α1β1 integrin protein expression in vivo, Supplemental FIG. 2, and therefore upregulated expression of the α1β1 integrin did not compensate for lack of the α2β1 integrin.

As endothelial cells are known to produce angiogenic growth factors, it was determined whether increased secretion of some of these factors might account for the increased α2-null endothelial cell proliferation. For this reason, levels of secreted VEGF and PLGF in conditioned media from wild type and α2-null endothelial cells were determined by ELISA. α2-null endothelial cells secreted slightly greater amounts of VEGF (116+/−21 pg/ml versus 74+/−17 pg/ml [p=O.15]) and PLGF (87+/−25 pg/ml versus 67+/−17 pg/ml [p=O.53]) than the wild type cells. However, the increased amount of VEGF and PLGF secreted by α2-null endothelial cells was not significantly different and it seemed that it alone could not have accounted for the dramatic alterations in cell proliferation that were observed.

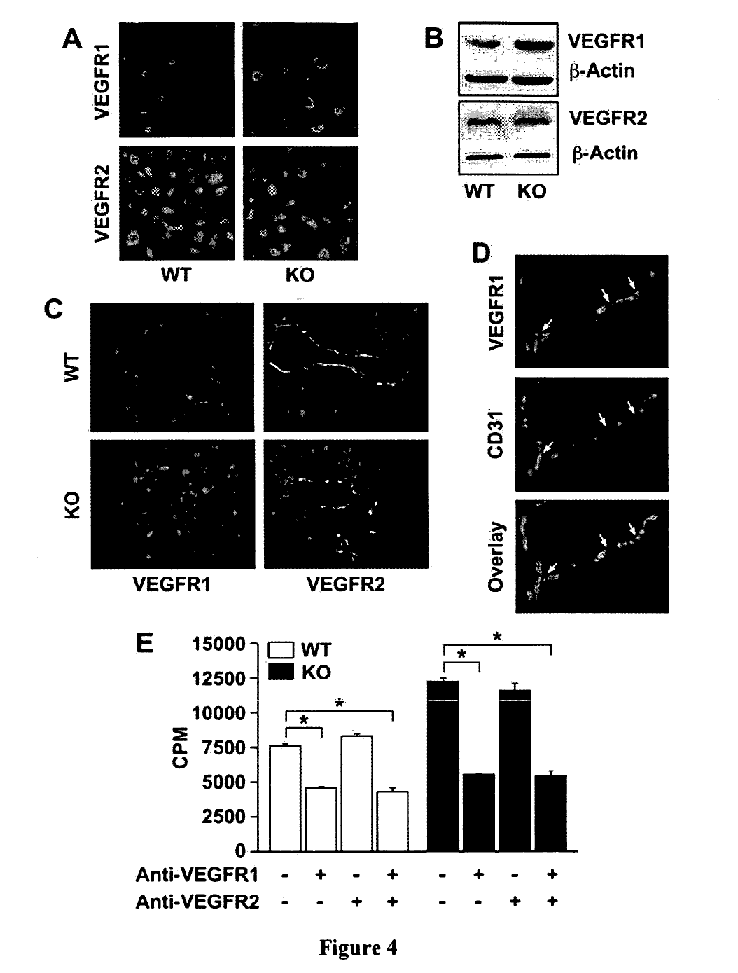

Increased production of angiogenic growth factors is not the only mechanism available to stimulate endothelial cell proliferation. Increased expression of angiogenic growth factor receptors such as VEGFR1 and VEGFR2 might also account for increased proliferation. Therefore, the levels of expression of VEGFR1 and VEGFR2 were determined by both immunofluorescence and Western blot analysis. Immunofluorescence and immunoblot analysis showed similar levels of VEGFR2 on both wild type and α2-null endothelial cells (FIGS. 4A and B). In contrast, VEGFR1 was expressed at higher levels by α2-null endothelial cells compared to wild type cells (FIGS. 4A and B). These data suggest that the increased proliferation observed in the α2-null primary pulmonary endothelial cells might be in part driven by increased expression of VEGFR1.

To determine whether tumor vessels in the α2-null mouse also expressed higher levels of VEGFR1, but not VEGFR2, compared to their wild type counterparts, tumor sections were co-stained with anti-VEGFR1 or anti-VEGR2 and anti-CD31 antibodies.

Similar to the in vitro data, endothelial cells within the tumors of α2-null, but not wild type mice expressed high levels of VEGFR1 (FIGS. 4C and D). In contrast, comparable levels of VEGFR2 were detected in tumor vessels of both wild type and α2-null mice (FIG. 4C). These findings suggest that, in vivo, the increased tumor angiogenesis and endothelial cell proliferation in the α2-null animals is in part due to increased expression of VEGFR1.

If increased levels of VEGFR1 expression in the α2-null primary endothelial cells contributed to the intrinsic proliferative potential, then inhibition of VEGFR1, but not VEGFR2, should abrogate the proliferative advantage. As shown in FIG. 3D and in FIG. 4E, the α2-null endothelial cells proliferated more rapidly than wild type endothelial cells.

As shown in FIG. 4E, incubation of cells with the anti-VEGFR1 inhibitory antibody significantly reduced proliferation of both the wild type and α2-null endothelial cells, but the effect was greater in the α2-null endothelial cells. Addition of the anti-VEGFR2 antibody failed to inhibit proliferation of either the α2-null or wild type endothelial cells in this system.

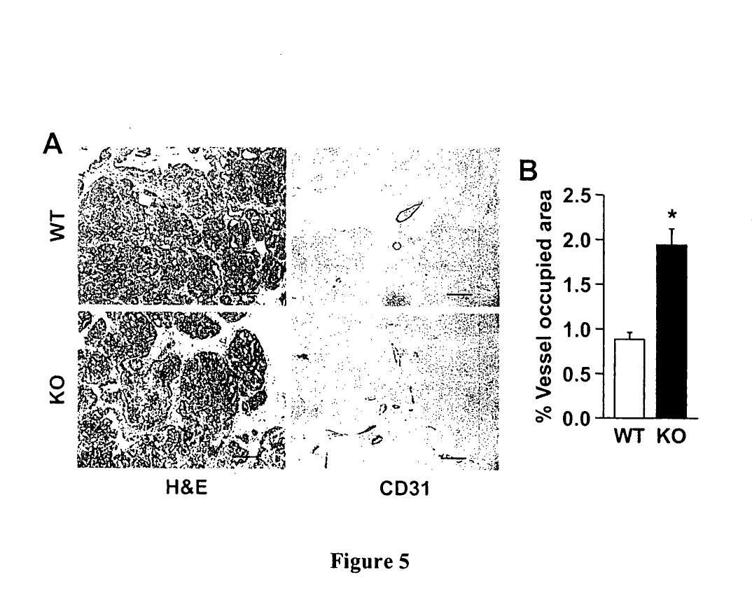

These observations appear to contradict earlier data that suggested that the α2β1 integrin was proangiogenic. Therefore, to insure that the marked increase in angiogenesis observed in the absence of the α2β1 integrin was not unique to a single tumor model, neoangiogenesis was compared in wild type and α2-null mice in another model. In spontaneous MMTV-PyMT oncogene-induced mammary cancer, tumor angiogenesis was significantly increased in α2-null tumors compared to tumors from wild type littermates (FIGS. 5A and B). Furthermore, it was also observed that during wound healing the α2-null mice show significantly increased angiogenesis within the granulation tissue at day 10 (data not shown).

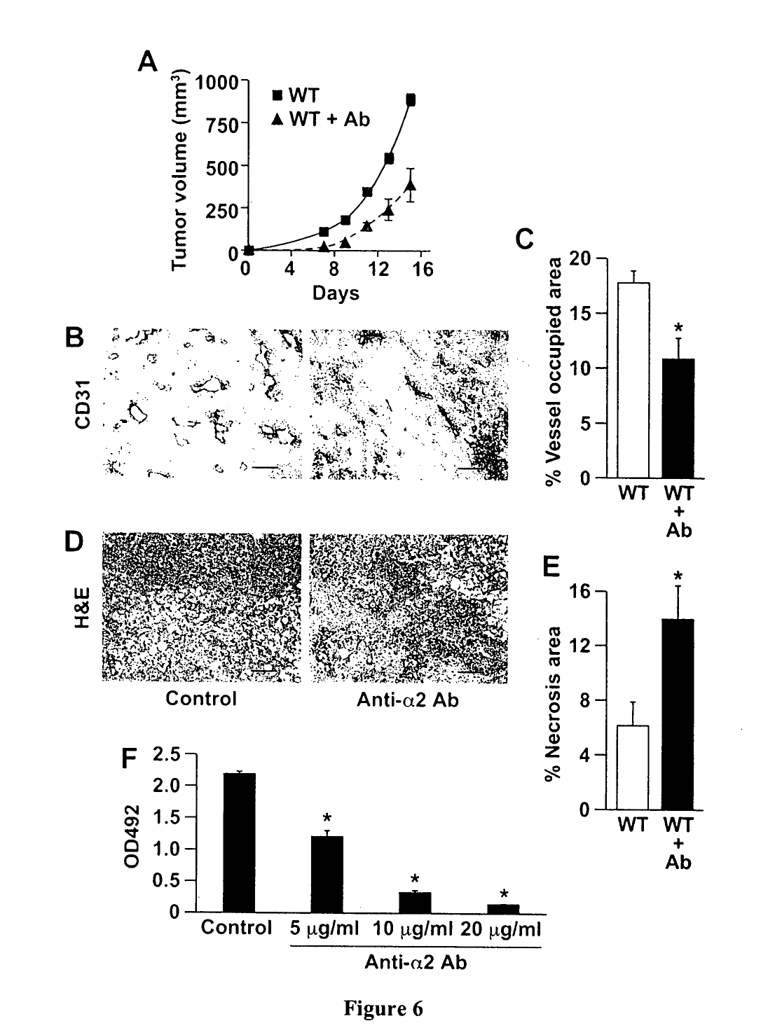

These results suggested a perplexing paradox in light of the previously published data using inhibitory antibodies [Senger D R, Claffey K P, Benes J E, Perruzzi C A, Sergiou A P, Detmar M: Angiogenesis promoted by vascular endothelial growth factor: regulation through alpha 1 beta1 and alpha2beta1 integrins. Proc Natl Acad Sci USA 1997, 94(25):13612-13617; Senger D R, Penuzzi C A, Streit M, Koteliansky V E, de Fougerolles A R, Detmar M: The alpha(1)beta(1) and alpha(2)beta(1) integrins provide critical support for vascular endothelial growth factor signaling, endothelial cell migration, and tumor angiogenesis. Am] Pathol 2002, 160(1): 195204; Whelan M C, Senger D R: Collagen I initiates endothelial cell morphogenesis by inducing actin polymerization through suppression of cyclic AMP and protein kinase A.] Biol Chem 2003, 278(1):327-334]. The inhibitory antibody data suggested that the α2β1 integrin plays a proangiogenic role. To address this conundrum, wild type and α2-null mice were injected subcutaneouly with B16F10 melanoma cells on day 0 in a manner identical to that described above. The animals were then injected intraperitoneally with an inhibitory anti-α2β1 integrin antibody or control anti-IgG antibody on days 2, 5, and 7. Palpable tumors were evaluated for size every two days for 14 days and tumor blood flow quantitated on days 7 and 14 by power Doppler. As shown in FIG. 6A, tumor size was significantly reduced in wild type mice, but not in α2-deficient mice receiving inhibitory anti-α2β1 Integrin antibody.

After 14 days the tumors were excised. The total vascular area in tumor sections from wild type mice treated with anti-α2β1 integrin was significantly decreased compared to wild type mice treated with control antibody (FIGS. 6B and C). As expected the extent of necrosis was significantly increased in wild type mice treated with anti-α2β1 integrin antibody (FIGS. 6D and E). These findings suggest that ligation of the α2β1 integrin with an inhibitory anti-α2β1 integrin antibody augments the antiangiogenic function of the integrin.

Since in vivo treatment of the inhibitory anti-α2β1 antibody dramatically altered tumor angiogenesis and vessel morphology, the effect of inhibitory anti-α2β1 integrin antibody on primary endothelial cell proliferation was evaluated. As shown in FIG. 6F, the addition of the inhibitory anti-α2β1 integrin antibody inhibited proliferation in a dose-dependent fashion. Although the antibody inhibited endothelial cell proliferation, the inhibitory anti-α2β1 integrin antibody did not stimulate apoptosis (data not shown). These data demonstrate that ligation of the α2β1 integrin by inhibitory antibody confers an antiproliferative signal, but not an apoptotic signal to endothelial cells.

The above demonstrates the importance of the α2β1 integrin in regulating pathologic angiogenesis and vascular morphogenesis is firmly established. Demonstrated here for the first time is the increased expression of the α2β1 integrin on tumor endothelium in comparison to quiescent endothelium. The in vivo data presented herein clearly demonstrate that mice lacking α2β1 integrin expression exhibit increased tumor growth and angiogenesis with decreased tumor necrosis. Also, enhanced tumor angiogenesis is associated with increased expression of VEGFR1 by endothelial cells both in vitro and in vivo. The finding that integrin α2-null mice challenged with B16 melanoma or with spontaneous mammary tumors shift the angiostatic balance in favor of angiogenesis, suggests for the first time that expression of the α2β1 integrin is anti-angiogenic in vivo. In addition, signals downstream of the α2β1 integrin regulate the quiescent vascular phenotype and modulate the recruitment of pericytes and normalization of vessel morphology. On the other hand, inhibitory anti-α2β1 integrin antibodies inhibit tumor growth, tumor angiogenesis, and promote tumor necrosis. Together the data suggest that signals downstream of α2β1 integrin expression and/or ligation are anti-angiogenic.

### Experimental Procedures

### Cell Lines, Mice, and Tumor Studies

α2 Integrin subunit-deficient mice were backcrossed between 8-10 times onto the C57jBL6 or FVB background to obtain animals that were 99% genetically C57jBL6 or FVB. The animals were housed in pathogen-free conditions at Vanderbilt University Medical Center, in compliance with IACUC regulations. All animals used for the B16F10 cells, a melanoma experiments were used at 6 to 12 weeks of age. Within individual experiments, mice were appropriately age and sex matched. B16F10 cells, a melanoma cell line derived from C57jBL6 mice, were maintained in DMEM supplemented with 10% FBS at 37° C. in a humidified CO2 (5%) atmosphere. Mice were injected subcutaneously into the flank with syngeneic, B16F10 melanoma cells (1×106) and monitored for palpable tumors every three days for 21 days. For antibody inhibition, the inhibitory anti-α2β1 integrin antibody, Ha1j29, (250 IJgjanimal) was injected intra peritoneally on day two after tumor cell implantation and then every three days until the end of the experiment. At each observation, the size of the tumor in the wild-type (n=9) versus the α2-deficient mice (n=9) was measured with calipers. Tumor volume was determined using the equation: volume=a×(b)2×0.52, where a is the longest dimension and b is the shortest. After 2 or 3 weeks, tumors were excised and a portion of each was placed in 10% formalin for paraffin embedding or snap-frozen in Tissue-Tek® a.C.T. Compound (Sakura Finetek U.S.A., Inc. Torrance, Calif.).

Wild type and α2 integrin subunit-deficient mice on the FVB background were crossed with MMTV-PyMT transgenic animals to obtain wild type transgene expressing and α2-null transgene expressing animals. Animals were sacrificed at 8 weeks of age and mammary glands were harvested.

Histology, Immunohistochemistry, and Immunofluorescence Analyses

Tumor morphology was initially evaluated on paraffin-embedded, hematoxylin and eosin stained sections. The area of tumor necrosis was determined on stained sections photographed at IOX. Necrotic areas were measured quantitatively in nine low power (IOX) fields for each tumor using the Metamorph imaging system. All immunohistochemical and immunofluorescence analyses were carried out on 5 μm frozen sections. For immunofluorescent staining, frozen sections were fixed sequentially in cold 100% acetone, acetone:chloroform (1 volume:1 volume), and 100% acetone and stained with anti-mouse CD31, anti-al integrin subunit (Clone Ha31/8), anti-α2 integrin subunit (Clone HM α2), anti-VEGFR1 (flt-1), anti-VEGFR2 (flk-1), anti-a or anti-Ki67. The signal was visualized with secondary antibodies, Alexa 594 or Alexa 488conjugated Ig and nuclei were visualized with DAPI. The slides were examined under fluorescent microscopy and pictures were processed with Metamorph imaging system.

For immunohistochemical visualization of CD31 staining, a biotin-conjugated secondary antibody and diaminobenzidine substrate were employed. The cross-sectional area of CD31-positive structures was determined quantitatively using NIH Image Analysis software (version 1.62; National Institutes of Health, Bethesda, Md.). Primary cells were grown on glass slides and fixed in 100% methanol prior to immunofluorescent staining.

### Fluorescence Angiography

Tumor vasculature was visualized by fluorescent angiography by using a TRITC-labeled Dextran injected retro-orbitally (5% in PB S, 100 μl/l) into mice. After 15 minutes, mice were sacrificed, tumors were frozen in OCT and the frozen section examined using fluorescence microscopy.

### Ultrasound Data Acquisition

A VisualSonics 770 high-resolution imaging system equipped with a 30 MHz transducer was used for these experiments. After anesthetization (2%/98% isofluorane/oxygen), the animal was restrained on a flat surface and the transducer was centered on the tumor and held in place by a stabilized holder. Coupling gel was applied over the entire tumor and was also applied to the transducer. Scout images were obtained to determine the extent of the tumor region via 3D B-mode imaging.

The transducer holder allows for the steady acquisition of a 512×512 acquisition matrix over a 12-15 mm field of view (depending on tumor size) and a 20 mm image width. During 3D acquisition, the holder gently and automatically slides over the length of the tumor and acquires one image at each of 90-170 (depending on tumor size) contiguous slices that are each 100 microns thick. Once the 3D B-mode acquisition was optimized to cover the whole length of the tumor, 3D power Doppler images were acquired with the same field of view and imaging dimensions. The scan speed and wall filters were held constant at 2.0 mm/s and 2.5 mm/s, respectively, for all studies. In power Doppler images, regions with blood flow are assigned a color level in arbitrary units from no power Doppler signal to maximum power Doppler signal. For each slice in the 3D power Doppler image stack, the fractional area displaying a power Doppler signal was calculated. By dividing the summed number of voxels displaying a power Doppler signal by the total tumor area, the percent vascularity was calculated.

### Immunoblot Analysis

Endothelial cells were washed twice with ice-cold PBS and lysed in lysis buffer (50 mM Tris-HCI with 1% sodium dodecyl sulfate and 1% B-mercaptoethanol, 10 μg/ml aprotinin, 5 pg/ml leupeptin, 40 mmol/L NaF, 0.5 mmol/L phenyl methyl sulfonyl fluoride, 0.5 mmol/L o-vanadate, and 1 mmol/L dithiothreitol). Total protein concentration was determined by the Pierce protein assay). Equivalent amounts of protein lysate were subjected to sodium dodecyl sulfate-polyacrylamide gel electrophoresis and electroblotted onto Immobilon-P transfer membrane. Immunoblots were incubated overnight with the appropriate dilution of primary antibody at 4° C. followed by secondary horseradish peroxidase-conjugated sheep anti-mouse or anti-rabbit antibody for 1 hour at room temperature. Enhanced chemiluminescence system was used for visualization.

### Recovery of Mouse Lung Endothelial Cells

Recovery of primary pulmonary endothelial cells was carried out essentially as described [Pozzi A, Moberg P E, Miles L A, Wagner S, Soloway P, Gardner HA: Elevated matrix metalloprotease and angiostatin levels in integrin alpha 1 knockout mice cause reduced tumor vascularization. Proc Nat/Acad Sci USA 2000, 97(5):2202-2207]. C57/BL6 wild-type and α2-null mice (2 months old) were anesthetized, and the lung vasculature was perfused with PBS/2 mM EDTA followed by 0.25% trypsin/2 mM EDTA via the right ventricle. Heart and lungs were removed en bloc and incubated at 37° C. for 20 min. The visceral pleura then was trimmed away, and the perfusion was repeated. Primary endothelial cells (>90% pure by immunostaining with anti-CD31) were recovered and grown on tissue culture plastic for 3 days in EGM-2-MV containing 5% FCS.

For proliferation assays, 5×103 primary endothelial cells were plated in EGM2-MV on 96-well plates coated with either 10 μg/ml collagen I, 10 μg/ml collagen IV, 10 μg/ml fibronectin or on tissue culture plastic. After 3 days, cells were pulsed for an additional 48 hr with 3H-thymidine (1 μCi/well). In the inhibition experiment, complete medium was changed to serum free medium after 12 hr. Neutralizing antibodies anti-flt-1 or anti-flk-1 (10 μg/ml) were added and replaced fresh every other day. After 3 days, cells were pulsed for an additionally 48 hours with 3H-thymidine. Proliferation assays using the inhibitory anti-α2β1 integrin antibody were performed using the CellTiter 96 Aqueous Non-radioactive Cell proliferation Assay Kit. The inhibitory anti-α2131 integrin antibody (clone Hal/29) (5 μg/ml, 10 μg/ml and 20 μg/ml) or isotype control IgG was added to 2×104 cells in a 96-well plate at 24 hours. After 48 hours, 201-11 combined MTS/PMS substrate was added into each well and the absorbance at 490 nm was record after 2 hour incubation at 37° C. in the incubator.

### Real-Time Quantitative RT-PCR

The mRNA levels of the integrin subunit genes in the wild type and α2-null mice were analyzed by real-time RT-PCR. Total RNA was isolated from endothelial cells using the Trizol@Reagent procedure, and further purified with RNeasy RNA extraction kit. The mRNAs were reverse-transcribed into cDNAs using iScript cDNA synthesis kit. Real-time amplifications were performed in 96-well plates in the Bio-Rad i-Cycler system. Each 25\-II amplification contained an aliquot of reverse transcription reaction, 1× iQ SYBR Green Supermix (Bio-Rad) and 0.21.lM of each forward and reverse primer for each selected gene. Fluorescence emission from the reactions was monitored in real time over a 40-cycle range with alternating denaturation (95° C. for 15 s) and annealing/extension (60° C. for 60 s) steps. The mRNA level for each gene was calculated using the relative standard curve method. Samples were normalized using GAPDH mRNA.

### Statistical Analysis

All experiments were repeated three or four times. Statistical analysis was performed using either ANOVA or unpaired student's t test and p<0.05 was considered statistically significant. All calculations and graphs were performed using GraphPad Prism Version 4.

### REFERENCES

1. Hanahan D, Weinberg R A: The hallmarks of cancer. Cell 2000, 100(1):5770.
2. Vogelstein B, Kinzler KW: Cancer genes and the pathways they control. Nat Med 2004, 10(8): 789-799.
3. DeClerck Y A, Mercurio A M, Stack M S, Chapman H A, Zutter M M, Muschel R J, Raz A, Matrisian L M, Sloane B F, Noel A et a/: Proteases, extracellular matrix, and cancer: a workshop of the path B study section. Am] Patho/2004, 164(4): 1131-1139.
4. Coussens L M, Fingleton B, Matrisian LM: Matrix metalloproteinase inhibitors and cancer: trials and tribulations. Science 2002, 295(5564): 2387-2392.
5. Matrisian L M, Sledge G W, Jr., Mohla S: Extracellular proteolysis and cancer: meeting summary and future directions. Cancer Res 2003, 63(19): 6105-6109.
6. Hanahan D, Lanzavecchia A, Mihich E: Fourteenth Annual Pezcoller Symposium: the novel dichotomy of immune interactions with tumors. Cancer Res 2003, 63(11):3005-3008.
7. Cunha G R, Matrisian LM: It's not my fault, blame it on my microenvironment. Differentiation 2002, 70(9-10):469-472.
8. Hynes R O: A reevaluation of integrins as regulators of angiogenesis. Nat Med 2002, 8(9):918-921.
9. Jain R K: Molecular regulation of vessel maturation. Nat Med 2003, 9(6): 685-693.
10. McDonald D M, Teicher B A, Stetier-Stevenson W, Ng S S, Figg W D, Folkman J, Hanahan D, Auerbach R, O'Reilly M, Herbst R et a/: Report from the society for biological therapy and vascular biology faculty of the NCI workshop on angiogenesis monitoring.] Immunother 2004, 27(2): 161175.
11. Kerbel R, Folkman J: Clinical translation of angiogenesis inhibitors. Nat Rev Cancer 2002, 2(10):727-739.
12. Brooks P C, Stromblad S, Sanders L C, von Schalscha T L, Aimes R T, StetlerStevenson W G, Quigley J P, Cheresh D A: Localization of matrix metalloproteinase MMP-2 to the surface of invasive cells by interaction with integrin alpha v beta 3. Ce1/1996, 85(5):683-693.
13. Brooks P C, Montgomery A M, Rosenfeld M, Reisfeld R A, Hu T, Klier G, Cheresh D A: Integrin alpha v beta 3 antagonists promote tumor regression by inducing apoptosis of angiogenic blood vessels. Ce1/1994, 79(7): 1157-1164.
14. Brooks P C, Clark R A, Cheresh D A: Requirement of vascular integrin alpha v beta 3 for angiogenesis. Science 1994, 264(5158):569-571.
15. Eliceiri B P, Cheresh D A: The role of alphav integrins during angiogenesis: insights into potential mechanisms of action and clinical development.] Clin Invest 1999, 103(9): 1227-1230.
16. Senger D R, Claffey K P, Benes J E, Perruzzi C A, Sergiou A P, Detmar M: Angiogenesis promoted by vascular endothelial growth factor: regulation through alpha1beta1 and alpha2beta1 integrins. Proc Natl Acad Sci USA 1997, 94(25):13612-13617.
17. Senger D R, Perruzzi C A, Streit M, Koteliansky V E, de Fougerolles A R, Detmar M: The alpha(1)beta(1) and alpha(2)beta(1) integrins provide critical support for vascular endothelial growth factor signaling, endothelial cell migration, and tumor angiogenesis. Am] Pathol 2002, 160(1): 195204.
18. Whelan M C, Senger D R: Collagen I initiates endothelial cell morphogenesis by inducing actin polymerization through suppression of cyclic AMP and protein kinase A.] Biol Chem 2003, 278(1):327-334.
19. Sweeney S M, Dilullo G, Slater S J, Martinez J, Iozzo R V, Iauer-Fields J I, Fields G B, San Antonio JD: Angiogenesis in collagen I requires alpha2beta1 ligation of a GFP*GER sequence and possibly p38 MAPK activation and focal adhesion disassembly.] Biol Chem 2003, 278(33):3051630524.
20. Hong Y K, lange-Asschenfeldt B, Velasco P, Hirakawa S, Kunstfeld R, Brown I F, Bohlen P, Senger D R, Detmar M: VEGF-A promotes tissue repairassociated lymphatic vessel formation via VEGFR-2 and the alpha1beta1 and alpha2beta1 integrins. FasebJ 2004, 18(10):11111113.
21. Liu Y, Senger D R: Matrix-specific activation of Src and Rho initiates capillary morphogenesis of endothelial cells. FasebJ 2004, 18(3):457-468.
22. Pozzi A, Moberg P E, Miles L A, Wagner S, Soloway P, Gardner HA: Elevated matrix metalloprotease and angiostatin levels in integrin alpha 1 knockout mice cause reduced tumor vascularization. Proc Nat/Acad Sci USA 2000, 97(5):2202-2207.
23. Pozzi A, Wary K K, Giancotti F G, Gardner HA: Integrin alpha1beta1 mediates a unique collagen-dependent proliferation pathway in vivo.) Cell Sio/1998, 142(2): 587-594.
24. Chen J, Diacovo T G, Grenache D G, Santoro S A, Zutter MM: The alpha(2) integrin subunit-deficient mouse: a multifaceted phenotype including defects of branching moiphogenesis and hemostasis. Am) Patho/2002, 161(1):337-344.
25. Shan S, Robson N O, Cao Y, Qiao T, Li C Y, Kontos C D, Garcia-Blanco M, Dewhirst M W: Responses of vascular endothelial cells to angiogenic signaling are important for tumor cell survival. FasebJ 2004, 18(2): 326-328.
26. Morikawa S, Baluk P, Kaidoh T, Haskell A, Jain R K, McDonald O M: Abnormalities in pericytes on blood vessels and endothelial sprouts in tumors. Am] Patho/2002, 160(3):985-1000.
27. Hobbs S K, Monsky W L, Yuan F, Roberts W G, Griffith L, Torchilin V P, Jain R K: Regulation of transport pathways in tumor vessels: role of tumor type and microenvironment. Proc Nat/Acad Sci USA 1998, 95(8):4607-4612.
28. Bergers G, Benjamin L E: Tumorigenesis and the angiogenic switch. Nat Rev Cancer 2003, 3(6):401-410.
29. Caimeliet P, Moons L, Luttun A, Vincenti V, Compernolle V, De Mol M, Wu Y, Bono F, Devy L, Beck H et at: Synergism between vascular endothelial growth factor and placental growth factor contributes to angiogenesis and plasma extravasation in pathological conditions. Nat Med 2001, 7(5): 575-583.
30. Luttun A, Tjwa M, Moons L, Wu Y, Angelillo-Scherrer A, Liao F, Nagy J A, Hooper A, Priller J, De Klerck B et a/: Revascularization of ischemic tissues by PIGF treatment, and inhibition of tumor angiogenesis, arthritis and atherosclerosis by anti-FIU. Nat Med 2002, 8(8):831-840.
31. Lyden D, Hattori K, Dias 5, Costa C, Blaikie P, Butros L, Chadburn A, Heissig B, Marks W, Witte Let a/: Impaired recruitment of bone-marrowderived endothelial and hematopoietic precursor cells blocks tumor angiogenesis and growth. Nat Med 2001, 7(11):1194-1201.
32. Hattori K, Heissig B, Wu Y, Dias 5, Tejada R, Ferris B, Hicklin D J, Zhu Z, Bohlen P, Witte Let a/: Placental growth factor reconstitutes hematopoiesis by recruiting VEGFR1 {+) stem cells from bone-marrow microenvironment. Nat Med 2002, 8(8):841-849.
33. Kaplan R N, Riba R D, Zacharoulis 5, Bramley A H, Vincent L, Costa C, MacDonald D D, Jin D K, Shido K, Kerns S A et a/: VEGFR1-positive haematopoietic bone marrow progenitors initiate the pre-metastatic niche. Nature 2005, 438(7069):820-827.
34. Bergers G, Song 5: The role of pericytes in blood-vessel formation and maintenance. Neuro-onco/2005, 7(4):452-464.
35. Foo 55, Turner C J, Adams 5, Compagni A, Aubyn D, Kogata N, Lindblom P, Shani M, Zicha D, Adams R H: Ephrin-B2 controls cell motility and adhesion during blood-vessel-wall assembly. Cell 2006, 124(1):161173.
36. Song S, Ewald A J, Stallcup W, Werb Z, Bergers G: PDGFRbeta+perivascular progenitor cells in tumours regulate pericyte differentiation and vascular survival. Nat Cell Biol 2005, 7(9):870-879.
37. Gutheil J C, Campbell T N, Pierce P R, Watkins J D, Huse W D, Bodkin D J, Cheresh D A: Targeted antiangiogenic therapy for cancer using Vitaxin: a humanized monoclonal antibody to the integrin alphavbeta3. C/in Cancer Res 2000, 6(8): 3056-3061.
38. Bader B L, Rayburn H, Crowley D, Hynes R O: Extensive vasculogenesis, angiogenesis, and organogenesis precede lethality in mice lacking all alpha v integrins. Cell 1998, 95(4):507-519.
39. Reynolds L E, Wyder L, Lively J C, Taverna D, Robinson S D, Huang X, Sheppard D, Hynes R O, Hodivala-Dilke K M: Enhanced pathological angiogenesis in mice lacking beta3 integrin or beta3 and betaS integrins. Nat Med 2002, 8(1):27-34.
40. Bredesen D E, Mehlen P, Rabizadeh S: Apoptosis and dependence receptors: a molecular basis for cellular addiction. Physiol Rev 2004, 84(2):411-430.
41. Stupack D G, Teitz T, Potter M D, Mikolon D, Houghton P J, Kidd V J, Lahti J M, Cheresh D A: Potentiation of neuroblastoma metastasis by loss of caspase-8. Nature 2006, 439(7072):95-99.
42. Stupack D G, Cheresh D A: Apoptotic cues from the extracellular matrix: regulators of angiogenesis. Oncogene 2003, 22(56):9022-9029.
43. Stupack D G, Cheresh D A: Get a ligand, get a life: integrins, signaling and cell survival.] Cell Sci 2002, 11 S(Pt 19):3729-3738.

### FIGURE LEGENDS

FIG. 1. Growth of syngeneic, B16F10 melanoma is enhanced in α2β1 integrin-deficient mice. A: Tumor volume in α2-null mice and their wild type littermates on a pure C57jIB6 background as a function of time over 21 days. B16F10 melanoma cells (1×106) were injected s.c. into the flank and tumor growth was quantitated every other day. The data are presented as the mean ±SEM (p<O.OOOl, statistical analysis by ANOVA). B: Close-up of tumors after 21 days (scale bar=5 mm). C: Representative sections of tumors from α2-null and wild type animals stained with H&E (scale bar=501 Jm) (Magnification 200×). 0: Immunohistochemical analysis with anti-CD31 staining of tumor sections from wild-type and α2-null animals demonstrated a significant increase in size and number of vessels (scale bar=501 Jm) (Magnification 200×). E: Total area occupied by CD31 positive structures representing tumor vascular area as a percentage of total tumor area.

Tumors from α2-null hosts show increased vascularity (*p<0.05). Data are presented as the mean and SEM (9 tumors per genotype from 3 separate experiments).

FIG. 2. Vascular perfusion and tumor necrosis in tumors in α2-null and wild type mice. A: Vessel wall permeability determined by TRITC-dextran perfusion. Wild type mice show significant leakage of TRITC-dextran in comparison to α2-null mice. B:

Immunofluorescent analysis of CD31 (red)-positive endothelial cells and a-SMA (green)-positive pericytes in the tumor tissue from wild type and α2-null mice. Few double positive vessels were observed in the wild type animals consistent with poor recruitment of pericytes to the vessel wall. In contrast, many double positive vessels were identified in the α2-null mouse. C: The percentage of double (CD31 plus aSMA)-positive vessels to total CD31 positive vessels was quantified and the data was present with mean±SME (*p<O.Ol). D: The amplitude of blood flow within the tumor was determined by power Doppler. There was a marked increase in blood flow to the tumor (as seen in yellow) in the α2-null mice in comparison to wild type mice at 14 days after tumor injection. Total blood flow to the tumor was 7.68+1.24% in α2-null mice and 2.5+/−0.43% in the wild type controls. E:Representative low power images of H&E stained sections demonstrate tumor necrosis (necrosis outlined in black) (scale bar=100\Jm) Magnification 100×. F:

The extent of tumor necrosis in wild type and α2β1 integrin-deficient mice was quantitated morphologically. Necrotic area is presented as a percentage of the total tumor area from 5 low power fields from 11 tumors per genotype (Magnification 1 OOX).

FIG. 3. α2β1 integrin expression and matrix-independent endothelial cell proliferation in vivo and in vitro. A: Immunofluorescent analysis demonstrated colocalization of α2β1 integlin (red) and CD31 (green) in tumor tissue of wild type mice. Nuclei are stained with DAPI (blue). The merged images demonstrate colocalization of the α2β1 integrin on tumor, but not resting endothelial cells in the wild type mouse. B: Immunofluorescent analysis of proliferation (anti-Ki67 [red]), endothelial cells (anti-CD31 [green]) and nuclei (DAPI [blue]). Ki67 positive, α2-null tumor endothelial cell nuclei or Ki67 negative, wild type tumor endothelial cell nuclei are indicated by arrows. C: The percentage of the Ki67 and CD31 double-positive cells in tumor tissue of wild type or α2-null mice was quantitated by counting the number of CD31 positive cells that were Ki67 positive or negative in 10 high power fields (400× maginification). The data are presented as mean ±SME (*p<0.01).

D: Primary pulmonary microvascular endothelial cells from wild type and α2-null animals were cultured in 96 well dishes coated with either type I collagen, type IV collagen, fibronectin, or BSA (10 ugjml of each) and pulsed with 3H-thymidine (lj. 1 Cijwell) for 48 hours. Trichloroacetic acid-precipitated lysates prepared and absolute cpm incorporated shown. Bars and errors indicate the mean and SEM (3 separate experiments performed in quadruplicate) (*p<O.Ol).

FIG. 4. VEGFR1 but not VEGFR2 expression is upregulated on α2-null endothelial cells in vitro and in vivo. A: Immunofluorecent analysis of primary pulmonary microvascular endothelial cells from wild type and α2-null animals for expression of VEGFR1 and VEGFR2. Nuclei were stained with DAPI (blue). B: The levels of VEGFR1 and VEGFR2 expression by primary pulmonary microvascular endothelial cells were evaluated by immunoblot analysis. 13-actin was used as loading control.

The results shown are representative of three separate experiments. C: Immunofluorecent analysis of VEGFR1 (red) or VEGFR2 (green) expression on the tumor endothelial cells in α2-null and wild type mice. Nuclei were stained with DAPI (blue). D: Immunofluorecent analysis demonstrates co-localization of CD31 (red) and VEGFR1 (green) in tumor cells in the α2-null mouse. E: Inhibition of VEGFR1 signaling reduced endothelial cell proliferation in vitro. Primary pulmonary microvascular endothelial cells from wild type and α2-null animals were cultured in 96-well dishes coated with BSA (10 ug/ml) and pulsed with 3H-thymidine (If.lCi/well) for 48 hours. Anti-VEGFR1 (10 IJg/ml) and/or anti-VEGFR2(10 IJg/ml) neutralizing antibodies were added 48 hours prior to the addition of 3H-thymidine, as designated.

Trichloroacetic acid-precipitated lysates were prepared and absolute cpm incorporated shown. Bars and errors indicate the mean and SEM (3 experiments were performed in quadruplicate) (*p<0.01).

FIG. 5. Tumor angiogenesis is increased in spontaneous MMTV-PyMT-induced mammary carcinomas arising in the α2β1 integrin deficient-mouse. A. Tumor vascularity of MMTV-PyMT-induced mammary carcinomas was determined with antiCD31 staining of mammary cancers from wild-type and α2-null animals. Tumor angiogenesis was increased in the α2-null mouse (scale bar=501 Jm) (Magnification 200×). B: Total area occupied by CD31 positive structures representing tumor vascular area as a percentage of total tumor area. Tumors from α2-null hosts show increased vascularity (*p<0.05). Data are presented as the mean and SEM (7 tumors per genotype).

FIG. 6. Angiogenesis is inhibited in wild type mice treated with an inhibitory anti α2β1 integrin antibody. A: Tumor volume in wild type mice injected with either an inhibitory anti-α2β1 integrin antibody or control antibody as a function of time over 14 days. B16F10 melanoma cells (1×106) were injected s.c. into the flank of wild type mice and the inhibitory anti-α2β1 integrin or control anti-IgG antibody was administered intraperitoneally on days 2, 5, and 7. Tumor growth was quantitated every other day. After 14 days the tumors were excised. The data are presented as the mean +/−SEM (p<O.Ol, statistical analysis by ANOVA). B: Immunohistochemical analysis with anti-CD31 staining of tumor sections from wildtype animals treated with inhibitory anti-α2β1 integrin antibody demonstrated a marked decrease in tumor vascularity (scale bar=501 Jm) (Magnification 200×). C: The total vascular area in tumor sections from wild type mice treated with anti-α2131 integrin was significantly decreased compared to wild type mice treated with control antibody. Tumors from wild type mice treated with inhibitory anti-α2131 integrin antibody showed decreased vascularity (*p<0.05). Data are presented as the mean +/−SEM (6 tumors per genotype from 3 separate experiments). 0:

Representative low power images of H&E stained sections of tumors from wild type animals treated with inhibitory anti-α2β1 integrin or control IgG antibody demonstrate increased necrosis in animals treated with the inhibitory antibody (scale bar=SOlJm) (Magnification 200×). E: The extent of tumor necrosis in mice treated with inhibitory anti-α2β1 or control antibody was quantitated morphologically.

Necrotic area is presented as a percentage of the total tumor area from 5 low power fields from 6 tumors per genotype (Magnification 100×) (*p<O.OS). F: Inhibitory anti-α2β1 integrin antibody blocks endothelial cell proliferation in vitro. Primary pulmonary microvascular endothelial cells from wild type animals were cultured in 96-well dishes and inhibitory anti-α2β1 integrin antibody was added 48 hours prior to the addition of MTSjPMS substrate. Absorbance at 490 nm was recorded after two hours of incubation. Bars and errors indicate the mean and SEM (2 experiments were performed in quadruplicate) (*p<0.0001).

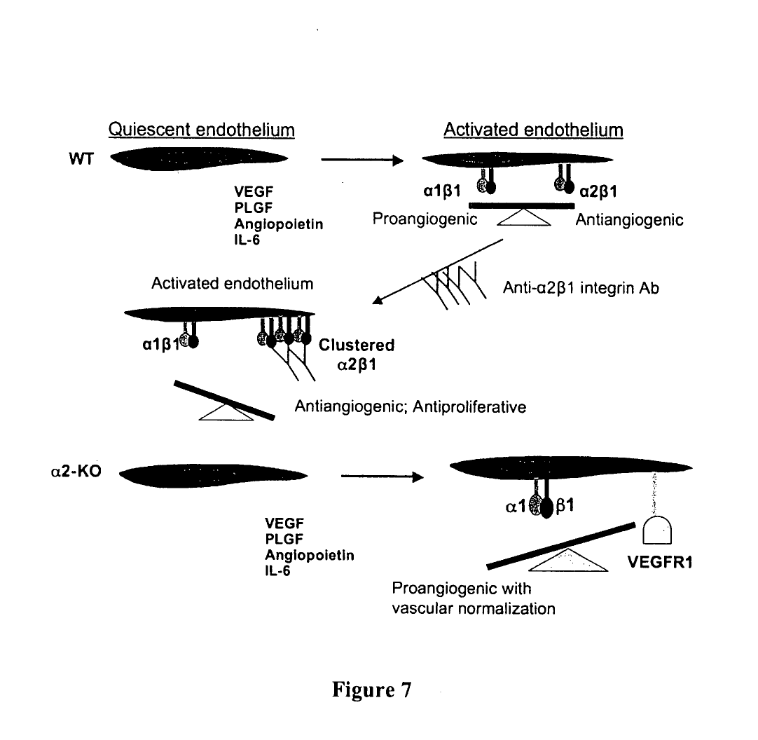

FIG. 7. A model of α2β1 integrin regulated-neoangiogenesis. Proposed herein is a model in which a balance between the α2β1 integrin and the α1β1 integrin is maintained to control the angiostatic set point. Neither the α2β1 or α1β1 integrin is required for developmental angiogenesis. In the resting state, endothelial cells express extremely low levels of both the α2β1 and the α1β1 integrin. However, under circumstances of pathologic angiogenesis, such as the tumor microenvironment, expressions of both the α2β1 and the α1β1 integrins is rapidly upregulated in wild type animals. These two integrins are not redundant but have distinct roles in angiogenesis. The α1β1 integrin provides pro-proliferative signals. In contrast, signals from the α2β1 integrin are anti-proliferative and serve to regulate vascular morphogenesis, suggesting that the two receptors serve a homeostatic balance. Part of the homeostatic balance involves down-regulation of the VEGFR1 expression by the α2β1 integrin. In the α2β1 integrin-deficient mouse, the α2β1 integrin-dependent anti-proliferative signals are released and VEGR1 is significantly upregulated on the tumor vessels but not other vessels within the animal. Neoangiogenesis is unchecked and vascular normalization occurs.

On the other hand, when inhibitory anti-α2β1 integrin antibodies are introduced into the system, the α2β1 integrin is ligated and antiproliferative signals emanating from the integrin are augmented leading to an inhibition of endothelial cell proliferation and a marked inhibition of angiogenesis.

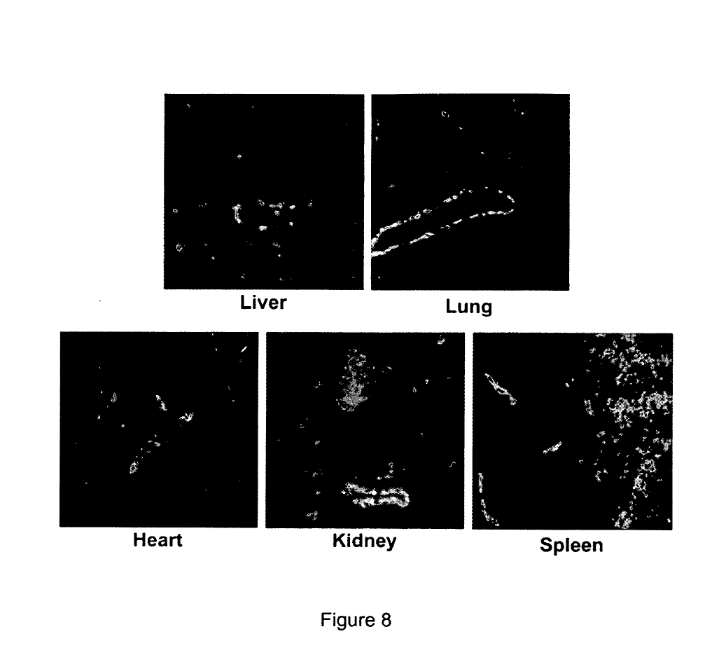

FIG. 8. Expression of the α2β1 integrin by resting and tumor vessels. A: Expression of the α2β1 integrin on quiescent endothelial cells in other tissues including the skin, heart, kidney, liver, and lung was not upregulated. Immunofluorescent analysis demonstrated co-localization of α2β1 integrin (red) and C031 (green) in tumor tissue, but not quiescent endothelial cells in tumor-bearing wild type mice. Nuclei are stained with OAPI (blue). The merged images fail to demonstrate co-localization of the α2β1 integrin with C031 on resting endothelial cells in the wild type mouse.

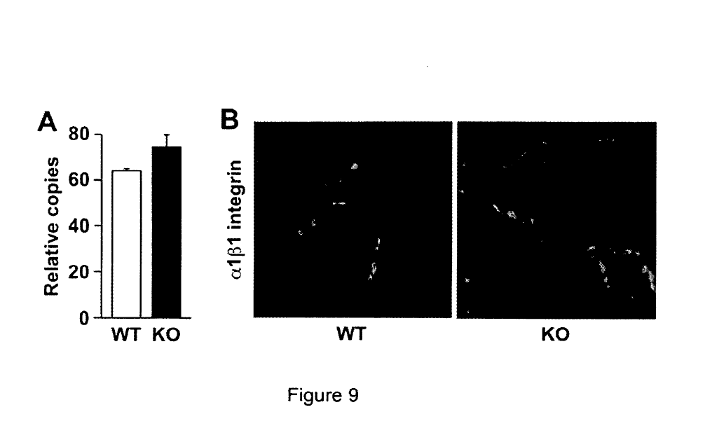

FIG. 9. Expression of the α1β1 integrin is not upregulated on α2-null endothelial cells in vitro or in vivo. A: Quantitative RT-PCR measurement of the mRNA level of the α1 integrin in the primary cultured endothelial cells from α2-null and wild type mice. B: Immunofluorecent staining of the α1 integrin on the tumor tissue. OAPI staining (blue) of the nuclei.

The above shows the importance of the α2 integrin in regulating pathologic angiogenesis and vascular morphogenesis is firmly established. It is demonstrated for the first time that increased expression of the α2 integrin on tumor endothelium in comparison to quiescent endothelium is antiproliferative. The in vivo data herein clearly demonstrate that mice lacking α2 integrin expression exhibit increased tumor growth and angiogenesis with decreased tumor necrosis. Enhanced tumor angiogenesis is associated with increased expression of VEGFR1 by endothelial cells both in vitro and in vivo. The finding that integrin α2-null mice challenged with B16 melanoma or with spontaneous mammary tumors shift the angiostatic balance in favor of angiogenesis, suggests for the first time that expression of the α2β1 integrin is anti-angiogenic in vivo. In addition, signals downstream of the α2β1 integrin regulate the quiescent vascular phenotype and modulate the recruitment of pericytes and normalization of vessel morphology. On the other hand, inhibitory anti-α2β1 integrin antibodies inhibit tumor growth, tumor angiogenesis, and promote tumor necrosis. Together the data suggest that signals downstream of α2β1 integrin expression and/or ligation are anti-angiogenic.

The studies in the α2β1 integrin-null mouse were initially surprising in light of the previously published data. In fact, Senger and colleagues showed that VEGF stimulates the expression of integrin α2β1 on human unbilical vein endothelial cells [16]. Inhibitory antibodies directed against the α1β1 and α2β1 integrins in combination inhibited adhesion of dermal microvascular endothelial cells, inhibited spreading of VEGF-stimulated cells on gels of polymerized type I collagen gels, inhibited endothelial cell haptotaxis and VEGF-stimulated chemotaxis, and prevented in vivo VEGF-induced angiogenesis in the cornea [16-18]. In addition, VEGFstimulated angiogenesis in mouse skin and angiogenesis into orthotopically implanted A431 squamous carcinoma cells in nude mice was blocked by inhibitory antibodies against the α2β1 integrin. Together these observations were interpreted to suggest that α2β1 integrin expression played a pro-angiogenic role. The present invention is predicated on the first experimental results defining the role of the α2β1 integrin in pathologic angiogenesis in animals lacking the α2β1 integrin. In addition to identifying a novel anti-angiogenic role for α2β1 integrin both in vitro and in vivo, these data clearly show potential limitations in drawing conclusions regarding signaling and regulatory pathways solely by use of inhibitory antibodies. The data herein suggest an alternative interpretation of the model and molecular mechanisms involved in α2β1 integrin-regulated angiogenesis.

To resolve the apparent contradiction between data generated in animals entirely lacking the α2β1 integrin and the earlier data generated using inhibitory antibodies, it is demonstrated that treatment of tumor-bearing wild type mice with inhibitory anti-α2β1 integrin antibodies resulted in even greater inhibition of angiogenesis and therefore even stronger anti-angiogenic signals both in vivo and in vitro. In addition, the inhibitory anti-α2β1 integrin antibody inhibits proliferation of wild type murine primary pulmonary endothelial cells, although cell survival was not affected.

Together these data suggest a model in which the α2β1 integrin sends an anti-proliferative signal to endothelial cells that tips the angiostatic set point in favor of anti-angiogenesis (FIG. 7). Release of the α2β1 integrin-mediated antiangiogenic signals in α2-null animals stimulates endothelial cell proliferation both in vivo and in vitro. The addition of inhibitory antibodies to wild type mice or endothelial cells augments the antiproliferative signals from the integrin.

The role of the VEGFR1 in tumor angiogenesis remains controversial. VEGFR1 can serve stimulatory as well as inhibitory roles in developmental angiogenesis. VEGFR1 is a high affinity receptor for VEGF, but also a specific receptor for placental growth factor (PLGF). PLGF is not essential during development but is required during pathologic angiogenesis [29, 30]. Over-expression of PLGF results in stabilized vessels suggesting that the control of vascular maturation by the α2β1 integrin may also be due to regulation (by the integrin) of VEGFR1 expression. Senger et al demonstrated that VEGF can upregulate expression of the both the α1β1 and α2β1 integrins [16], but cooperation between PLGF and integrins had not been studied. In many studies endothelial cells have not been found to express VEGFR1, but other cells including monocytes/macrophages and circulating endothelial progenitors have been observed to express VEGFR1 [31-33]. In the research leading to the present, demonstrated the expression of VEGFR1 by α2β1 integrin-null CD31 positive cells was demonstrated. The vessels in the α2-deficient mouse were increased in size and number, and occupied a greater area of the tumor. The vessels also showed improved vascular stability and decreased leakiness due to enhanced recruitment of pericytes to the vessel wall, all aspects of vascular normalization. The mechanisms that lead to the changes in the vessel shape and the vascular recruitment are an area of active investigation. The importance of the platelet derived growth factor and its receptor, Tie2-angiopoietin 1, and ephins and their ligands in vessel wall assembly are documented [9, 34-36]. This is the first report indicating a role for the integrins in not only regulating endothelial proliferation and survival, but also in controlling vessel patterning.

As mentioned earlier, genetic deletion of the α1β1 integrin supported the concept that the α1β1 integrin was pro-angiogenic [22]. Consistent with the proangiogenic role of the α1β1 integrin, mice in which the α1β1 integrin was genetically deleted demonstrated decreased tumor growth and decreased tumor angiogenesis. One possible explanation for the findings observed in the α2-null mouse would be compensatory up-regulation of the α1β1 integrin in the absence of the α2β1 integrin.

This possibility is excluded by the results of thr research leading to the present invention. In fact the levels of αβ1 integrin subunit mRNA as determined by qRT-PCR and α1β1 integrin protein as determined by immunofluorescence analysis were similar in vessels of the wild type and α2β1 integrin-deficient animals.

The data we report seem at first glance similar to the controversial findings regarding the av˜3 and av˜5 integrins. Based on the finding that integrin av˜3 is highly expressed in tumor vessels, Brooks, et al. demonstrated that inhibitory monoclonal antibodies directed against the av˜3 and av˜5 integrins, as well as peptides containing the RGD sequence, blocked angiogenesis in vivo and in vitro.

These data suggested a pro-angiogenic role for the av integrins [13, 14] and led to the development of a humanized inhibitory anti-av integrin antibody that is now in clinical trials [8, 10, 11, 37].

In striking contrast, the development of genetically-engineered mice lacking the integrin av subunit challenged the interpretation that the av integrins were proangiogenic. Genetic ablation of the av subunit did not alter embryonic vasculogenesis or angiogenesis, except for some changes in the cerebral microvessels [8, 38, 39]. Even more surprising was the observation that mice lacking one or both of av integrins showed enhanced tumor growth and tumor angiogenesis compared to wild type mice. The enhanced angiogenesis was found to be due, in part, to increased VEGFR2 expression on endothelial cells [39]. Vascular morphology was not addressed in detail. However, it appears that the increased expression of VEGFR2 and absence of av favored a less stabilized and quiescent vasculature.

The apparently discordant results obtained with the α2-null mouse and those obtained with inhibitory α2-integrin resemble in several, but not all, aspects the discordant roles reported for the av integrins in tumor angiogenesis. First, α2β1 integrin expression is significantly upregulated in tumor vessels. Second, genetic ablation of the α2 subunit, like ablation of the av, ˜3, or ˜5 integrin subunits failed to cause significant vascular abnormalities during development. Third, genetic ablation of these integrins resulted in enhanced tumor growth and tumor angiogenesis. Finally, genetic ablation of the α2 integrin subunit resulted in upregulation of VEGFR1.

Although there are similarities, the molecular mechanisms that underlie the α2β1 integrin and the av integrin paradoxes are different. First, as shown in FIG. 7, our model suggests that a balance between the contributions of α2β1 integrin and the al˜l integrin maintain and control the angiostatic set point. Neither the α1β1 nor the α2β1 integrin is required for developmental angiogenesis. In the resting state, endothelial cells express extremely low levels of the α1β1 and α2β1 integrins. However, under circumstances of pathologic angiogenesis, such as the tumor microenvironment or wound healing, expression of both the α1β1 and the α2β1 integrins is rapidly upregulated in normal, ie wild type, animals. The two integrins are not redundant but have distinct roles in angiogenesis. The α1β1 integrin provides proproliferative signals. In contrast, signals from the α2β1 integrin are anti proliferative, suggesting that the two receptors contribute to a homeostatic balance. Part of the homeostatic balance involves downregulation of the VEGFR1 expression by the α2β1 integrin. When inhibitory anti-α2β1 integrin antibodies are introduced into the system, the α2β1 integrin is clustered and anti proliferative signals are augmented leading to an inhibition of endothelial cell proliferation and a marked inhibition of angiogenesis. In the α2β1 integrin-deficient mouse, the α2β1 integrin-dependent anti-proliferative signals are released and VEGR1 is significantly upregulated on the tumor vessels, but not on other vessels within the animal. Neoangiogenesis and al˜l integrin-stimulated proliferative signals are unchecked.

An alternative model invokes the process termed integrin-mediated death (IMD) [40-43]. IMD occurs when cells that express a specific integrin are present in an inappropriate matrix that does not allow for integrin ligation. Unligated or antagonized, ie, antibody inhibited, integrin then recruits caspase-8 and stimulates apoptosis. IMD has been suggested to be the mechanism by which the inhibitory antibodies and peptides to the ov133 and ov135 stimulate endothelial cell apoptosis and inhibit angiogenesis, as well as to function in preventing metastasis of neuroblastoma via the α1β1 integrin, a recent manuscript published in Nature, January 2006. By invoking this process, we would postulate that in the normal, wild type animal under conditions of pathologic angiogenesis ligated α2β1 integrin prevents apoptosis. In addition, expression of the α2β1 integrin inhibits proliferation and balances the proangiogenic signals downstream of the α1β1 integrin. In the absence of α2 β1 integrin, compensatory upregulation of VEGFR1 results in enhanced endothelial proliferation. In the presence of the inhibitory antibody, the integrin is unligated and signals endothelial cell apoptosis.

So, why is the α2β1 integrin expressed? Does it serve a role other than in inhibiting signals from the α1β1 integrin? We would suggest that indeed it does. Beautiful in vitro data from Senger's group suggest that the role of the α2β1 integrin is in endothelial cell morphogenesis and tube formation. In fact, in the absence of the α2β1 integrin vascular morphology is dramatically altered with much larger lumens and many fewer tubules and a stabilized, well-perfused vascular bed. In our in vivo studies the α2β1 integrin controls not only endothelial cell proliferation, but also controls vessel morphogenesis, pericyte recruitment and vascular normalization.

In summary, the data presented here clearly demonstrate that mice lacking α2β1 integrin expression exhibit increased tumor angiogenesis associated with increased tumor growth and decreased tumor necrosis. Therefore, animals lacking the α2β1 integrin when challenged with B16 melanoma or when spontaneously developing breast cancer switch the angiostatic balance in favor of angiogenesis. The angiogenic switch in the α2-null mice results at least in part from an intrinsic proliferative advantage due to increased expression of the VEGFR1 on the α2-null endothelial cells both in vivo and in culture. Lack of the α2β1 integrin, combined with increased expression of VEGFR1 controls vascular stability. The contributions of VEGFR1 to vessel morphology in our model still must be defined. In wild type animals, the addition of inhibitory anti-α2β1 integrin antibodies inhibits tumor angiogenesis and leads to tumor necrosis by augmented anti-proliferative signals downstream of integrin ligation. Since the α2β1 integrin is only expressed by activated endothelial cells, inhibitory anti-α2β1 integrin antibodies provide a novel therapeutic target either alone or in combination with the anti-av integrin antagonists already in clinical trials.

Formulations of the antibody for therapeutic administration are prepared by mixing the antibody having the desired degree of purity with pharmaceutically acceptable carriers, diluents, excipients or stabilizers (Remington's Pharmaceutical Sciences 16th edition, Osol, A. Ed. (1980)), in the form of lyophilized formulations or aqueous solutions. Acceptable carriers, diluents, excipients, or stabilizers are nontoxic to recipients at the dosages and concentrations employed, and include buffers; preservatives; chelating agents; sugars; salt-forming counter-ions; metal complexes; and/or non-ionic surfactants.

The antibody is administered by any suitable means, including parenteral, subcutaneous, intraperitoneal, intrapulmonary, or intranasal. If desired for local immunosuppressive treatment, intralesional administration of the antibody is done. Parenteral administration includes intramuscular, intravenous, intraarterial, intraperitoneal, or subcutaneous administration. In addition, the antibody is suitably administered by pulse infusion, for example, with declining doses of the antibody. Preferably the dosing is given by injections, most preferably intravenous or subcutaneous injections.

For the prevention or treatment of disease, the appropriate dosage of antibody will depend on the type of disease to be treated, as defined above, the severity and course of the disease, whether the anti-.alpha.2 integrin antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease [from about 1 μg/kg to about 50 mg/kg] of antibody is a suitable dosage for administration to the subject, whether, for example, by one or more separate administrations, or by continuous infusion. A typical daily dosage might range [from about 1 μg/kg to about 50 mg/kg] or more, depending on the factors mentioned above. For repeated administrations over several days or longer, depending on the condition, the treatment is sustained until a desired suppression of disease symptoms occurs. However, other dosage regimens may be useful. The progress of this therapy is readily monitored by those skilled in the art. An antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, results from pharmacological and toxicity studies and other factors known to medical practitioners. A therapeutically effective amount of the antibody to be administered is determined by consideration of such, and is the minimum amount necessary to prevent, ameliorate, or treat the disorder. Such amount is preferably below the amount that is toxic to the host or renders the host significantly more susceptible to infections.

1. A method of treating an integrin-mediated disorder in a subject in need thereof comprising administering to the subject a therapeutically effective amount of at least one inhibitory anti-α2β1 integrin antibody.

2. The method of claim 1 wherein said integrin-mediated disorder is a α2β1 integrin-associated disorder.

3. The method of claim 1 wherein the integrin-mediated disorder is selected from the group consisting of restenosis, unstable angina, thromboembolic disorders, vascular injury or disease, atherosclerosis, arterial thrombosis, venous thrombosis, vaso-occlusive disorders, acute myocardial infarction, re-occlusion following thrombolytic therapy, re-occlusion following angioplasty, inflammation, rheumatoid arthritis, osteoporosis, bone resorption disorders, cancer, tumor growth, angiogenesis, multiple sclerosis, neurological disorders, asthma, macular degeneration, diabetic complications, diabetic retinopathy. inflammatory disease, autoimmune disease, Crohn's disease, ulcerative colitis, reactions to transplant, optical neuritis, spinal cord trauma, systemic lupus erythematosus (SLE), diabetes mellitus, Reynaud's syndrome, experimental autoimmune encephalomyelitis, Sjorgen's syndrome, scleroderma, juvenile onset diabetes, psoriasis, and infections that induce an inflammatory response.

4. The method of claim 1 wherein the therapeutically effective amount is between about 0.01 mg/kg/day and about 300 mg/kg/day.

5. A pharmaceutical composition comprising at least one inhibitory anti-α2β1 integrin antibody and a pharmaceutically acceptable carrier.

6. A method for treating a disease associated with abnormal angiogenesis, comprising administering to a subject in need thereof a therapeutically effective amount of at least one inhibitory anti-α2β1 integrin antibody.

7. The method of claim 6, wherein the disease associated with abnormal angiogenesis is a benign tumor or cancer.

8. The method of claim 7, wherein the benign tumor is selected from the group consisting of hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

9. The method of claim 7, wherein the cancer is selected from the group consisting of leukemia, breast cancer, skin cancer, bone cancer, prostate cancer, liver cancer, lung cancer, brain cancer, cancer of the larynx, gallbladder, pancreas, rectum, parathyloid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia Vera, adenocarcinoma, glioblastoma multiforma, lymphomas, malignant melanomas, epidermoid carcinomas, and other carcinomas and sarcomas.

10. The method of claim 6, wherein the disease associated with abnormal angiogenesis is selected from the group consisting of restenosis, atherosclerosis, insults to body tissue due to surgery, abnormal wound healing, diseases that produce fibrosis of tissue, repetitive motion disorders, disorders of tissues that are not highly vascularized, and proliferative responses associated with organ transplants.

11. The use of inhibitory anti-α2β1 integrin antibodies to inhibit tumor neoangiogenesis, slow tumor growth, treat abnormal angiogenesis, treat integrin-mediated disorders and inhibit endothelial cell proliferation.

12. An article of manufacture comprising packaging material and a pharmaceutical agent contained within said packaging material, wherein said pharmaceutical agent is effective for the treatment of a subject suffering from tumor neoangiogenesis, tumor growth, abnormal angiogenesis, integrin-mediated disorders and endothelial cell proliferation and wherein said packaging material comprises a label which indicates that said pharmaceutical agent can be used for ameliorating the symptoms associated therewith.
